(19) United States
(12) Patent Application Publication
(10) Pub. No.: US 2023/0386033 A1
(43) Pub. Date: Nov. 30, 2023

(54) Image Processing Apparatus, Image Processing Method, and Program

(71) Applicant: Hitachi, Ltd., Chiyoda-ku, Tokyo (JP)

(72) Inventors: Hanae YOSHIDA, Tokyo (JP); Masahiro OGINO, Tokyo (JP)

(21) Appl. No.: 18/031,675

(22) PCT Filed: Nov. 26, 2021
(86) PCT No.: PCT/JP2021/043449

§ 371 Date: Apr. 13, 2023

(30) Foreign Application Priority Data
Feb. 5, 2021 (JP) 2021-017690

(51) Int. Cl. G06T 7/00; G06T 7/13

(52) U.S. Cl. CPC G06T 7/0014; G06T 7/13; G06T 2207/20081; G06T 2207/20084; G06T 2207/30096

(57) ABSTRACT

Provided are a medical image processing apparatus and a medical image processing method capable of implementing specialized learning with higher accuracy in a case where the specialized learning is performed on a plurality of conventional features based on knowledge of a doctor. An image processing apparatus according to the present invention includes: an image group conversion unit that calculates a value of a predetermined feature (first feature) for each image constituting an input first image group, selects an image from the first image group on the basis of the value of the feature, and sets the image as an image of a second image group; and a feature extraction unit that extracts a new feature (second feature) by performing learning on the second image group generated by the image group conversion unit using a feature generation network.

### TECHNICAL FIELD

The present invention relates to an image processing technology used in a diagnosis support apparatus or the like that receives a medical image to be diagnosed and outputs a prediction result regarding diagnosis, and particularly relates to an image processing technology using machine learning.

### BACKGROUND ART

In diagnosis using a medical image inspection apparatus represented by an X-ray computed tomography (CT) apparatus, a magnetic resonance imaging (MRI) apparatus, or the like, it is common to reconstruct a captured three-dimensional medical image as a continuous two-dimensional cross section for observation and interpretation of the two-dimensional cross-sectional image. In the image interpretation, for example, shadow/shade detection, size measurement, determination of whether a shadow or a shade is normal or abnormal, determination of a lesion type of an abnormal shadow or shade, and the like are performed, and image features obtained in the process are used as auxiliary information when a doctor selects an optimal treatment method.

In recent years, the three-dimensional resolution of a medical image to be generated is also improved due to the advancement of an imaging apparatus, and data sizes tend to be larger. As a result, the generation interval of a two-dimensional cross section can be made shorter, and a lesion appearing on a medical image can be observed in more detail. However, as a result, the number of images per three-dimensional medical image increases, and the burden of interpretation also increases. In order to reduce a burden on a doctor and a technician when an enormous three-dimensional medical image is interpreted, a technology for automatically or semi-automatically implementing the above-described interpretation by applying an image processing technology using a computer has been developed. In the development, carrying out evidence-based medical care is an issue.

As a technology in which the image processing technology is applied to medical care, there is a method of assisting determination of a disease state, prognostic prediction, and selection of an optimal treatment method mainly from a radiological medical image by a discrimination model generated using various feature groups (hereinafter, a feature included in the above-described feature groups is referred to as a conventional feature) such as an average, variance, and shape index of pixel values. Features extracted by this method include a feature designed and evaluated by an expert, and can be said to reflect knowledge of a doctor.

On the other hand, a method of using a feature generated using deep learning as a substitute for a conventional feature or using the generated feature in combination with the conventional feature is also being developed, and there is a research report that the accuracy of prediction exceeds accuracy in a case where prediction is performed using only the conventional feature by these methods. However, it is known that a large data set is generally required for appropriate application of deep learning, and a feature is automatically extracted only from an input image, and thus, there is a case where a feature on which the doctor places importance is not incorporated.

In order to solve this problem and to achieve both improvement in a sense of satisfaction of a doctor and improvement in accuracy, some methods have been proposed in which both advantages of the conventional feature and the deep learning feature are utilized to fuse these features. For example, PTL 1 proposes a method of performing deep learning (hereinafter, referred to as specialized learning) specialized in a plurality of conventional features to calculate deep learning features specialized in the respective conventional features and then integrating the deep learning features. In PTL 1, when an input image according to a plurality of conventional features is generated, an image subjected to enhancement processing such as region segmentation or specific filter processing is used as learning data of deep learning, and specialized learning for some conventional features is implemented. In addition, PTL 2 discloses a method of classifying a medical image into any of a plurality of predetermined classes, and selecting an optimum restorer from a plurality of restorers corresponding to each of the plurality of classes according to the classification result.

### CITATION LIST

### Patent Literature

PTL 1: JP 2015-168404 A
PTL 2: JP 2019-25044 A

### SUMMARY OF INVENTION

### Technical Problem

Among conventional features, there is a conventional feature for which sufficient specialized learning cannot be implemented only by enhancement processing used in PTL 1 or the like or clustering performed on input data in a feature space in PTL 2.

For example, as one of important features in the case of determining the malignancy of a lung tumor from a chest CT image, there is a degree (degree of spiculation) of a spicula of the tumor contour. This is based on the knowledge that there is often a positive correlation between the degree of spiculation and the degree of malignancy. However, the degree of malignancy of a tumor is not defined only by this degree of spiculation, and this degree of spiculation also includes a doctor's sentimental judgment, and thus it is difficult to completely quantify the degree of spiculation only from an image. In order to incorporate this knowledge into machine learning, it is necessary to design a learning system that focuses on learning specialized for the degree of spiculation, that is, the relationship between the difference in the degree of spiculation and the difference in the degree of malignancy of the tumor. However, it is difficult to perform learning specialized for the degree of spiculation by a method proposed in the conventional techniques for the following reasons.

In the method of PTL 1 and the like, as means for causing learning specialized for a certain feature, a learning image is obtained by performing filter processing for enhancing a feature in an input image. However, the filter processing uniformly produces an effect on the entire image, and it is not possible to enhance only the height level of the degree of spiculation of the tumor contour. In addition, it is also difficult to perform clustering on the degree of spiculation that is difficult to quantify in a feature space.

An object of the present invention is to provide a medical image processing technology capable of implementing specialized learning with higher accuracy in a case where the specialized learning is performed on a plurality of conventional features based on knowledge of a doctor, and to enable the specialized learning even on the conventional features for which the specialized learning is difficult in the conventional method.

### Solution to Problem

In order to solve the above problems, the present invention calculates a feature of an input image group (first image group) for each of a plurality of features, creates a plurality of image groups (second image groups) from the input image group using the feature (first feature), and calculates and integrates new features (second features) for the plurality of image groups. The plurality of image groups are created based on a threshold for the first feature using a machine learning discriminator. The threshold for the first feature is set on the basis of a result of evaluating the identification performance of the discriminator on the basis of an objective variable of the discriminator.

That is, an image processing apparatus according to the present invention includes: an image group conversion unit that calculates a value of a predetermined feature (first feature) for each image constituting an input first image group, selects an image from the first image group on the basis of the value of the feature, and sets the image as an image of a second image group; and a feature extraction unit that extracts a new feature (second feature) by performing learning on each second image group generated by the image group conversion unit using a feature generation network.

An image processing method of the present invention includes: a step of inputting a first image group and calculating a value of a predetermined feature (first feature) for each image constituting the input first image group; a step of setting a threshold for the feature; an image group conversion step of selecting an image from the first image group on the basis of the value of the feature and setting the image as an image of a second image group; and a step of extracting a new feature (second feature) by performing learning on each second image group using a feature generation network. In the step of setting the threshold, a machine learning discriminator is generated for a predetermined feature, identification performance of the discriminator is evaluated, and the feature threshold for selecting the image is set based on the identification performance.

Furthermore, an image processing program of the present invention is a program that causes a computer to execute the method described above.

### Advantageous Effects of Invention

According to the present invention, it is possible to perform highly accurate specialized learning on a conventional feature while utilizing a feature of the conventional feature based on knowledge of a doctor without using a filter or the like. As a result, it is possible to improve both the satisfaction of the doctor and the accuracy, and to provide prediction information useful for the support of diagnosis and treatment.

### BRIEF DESCRIPTION OF DRAWINGS

FIG. 1 is a diagram illustrating a system configuration example including a medical image processing apparatus.

FIGS. 2(A) and 2(B) are diagrams each illustrating a configuration example of a part of the system illustrated in FIG. 1.

FIG. 3 is a flowchart illustrating an example of an outline of a process of the image processing apparatus illustrated in FIG. 1.

FIG. 4 is a flowchart illustrating a specific example of an image group conversion process according to a first embodiment.

FIG. 5 is a diagram illustrating an outline of the process according to the first embodiment.

FIG. 6 is a diagram illustrating an example of a second medical image group created in a medical image group conversion unit 21.

FIG. 7 is a diagram illustrating another example of the second medical image group created in the medical image group conversion unit 21.

FIG. 8 is a diagram illustrating an outline of processing according to a second embodiment.

FIG. 9 is a flowchart illustrating a feature selection process according to the second embodiment.

### DESCRIPTION OF EMBODIMENTS

Hereinafter, embodiments of the present invention will be described with reference to the drawings.

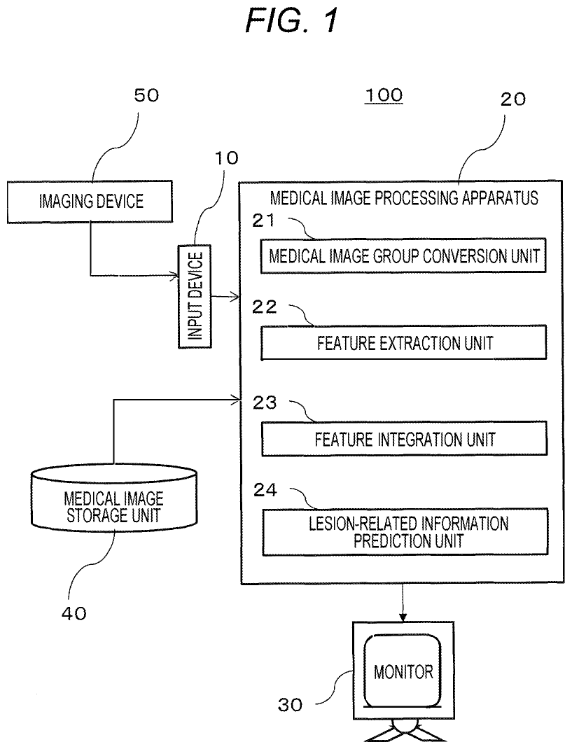

FIG. 1 is a diagram illustrating an overall configuration of a medical image processing system 100 to which the present invention is applied. As illustrated in FIG. 1, the medical image processing system 100 includes a medical image processing apparatus (hereinafter, simply referred to as an image processing apparatus) 20, an input device 10 that receives an operator's input or the like and transmits the input or the like to the image processing apparatus 20, and a monitor 30 that displays a prediction result such as a position, a size, a disease state, or the like of a lesion region or a specific organ obtained from the image processing apparatus 20, and a medical image or a part of the medical image. The image processing apparatus 20 has a storage device (image storage unit) in which a large number of medical images are stored inside or outside.

Here, the “lesion region” refers to a point and a region with a high suspicion of lesion that are determined on the basis of medical knowledge of a radiologist, medical basis (evidence) for the diagnosis of the disease, and the like. In a case where a lesion appears on a medical image, there is a high possibility that the lesion can be determined from a difference in luminance or a difference in distribution from the surroundings, that is, a region with a low suspicion of lesion, and the region is automatically or manually designated.

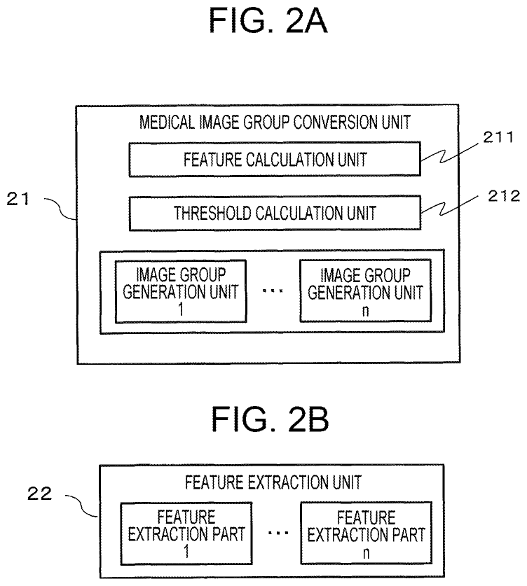

The image processing apparatus 20 includes a medical image group conversion unit 21 that generates a plurality of image groups (second image groups) from an input medical image group (a large number of medical images: a first image group), a feature extraction unit 22 that extracts a new feature (second feature) from each of the plurality of image groups, a feature integration unit 23 that integrates the new features, and a lesion-related information prediction unit (hereinafter, also simply referred to as a prediction unit) 24 that predicts a malignancy degree of a lesion, prognosis of a patient, and the like from the integrated features (hereinafter, integrated features). As illustrated in FIGS. 2(A) and 2(B), each of the medical image group conversion unit 21 and the feature extraction unit 22 includes image group generation units 1 to n and feature extraction units 1 to n as many as the number of conventional features used for the generation of the image groups.

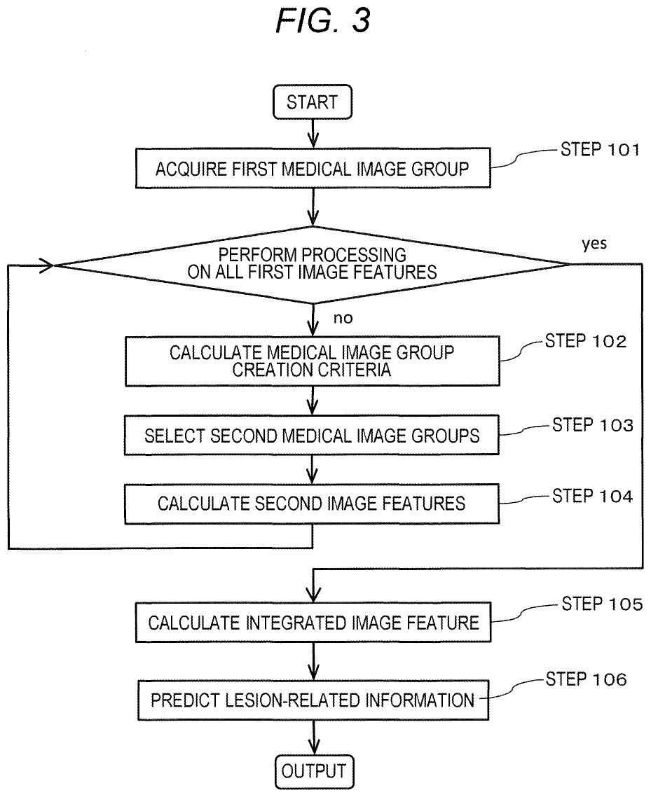

Next, an example of a process by the image processing apparatus 20 having the above system configuration will be described with reference to FIG. 3. The process by the image processing apparatus 20 is roughly divided into a process of generating a learning model and an operation process of applying a trained learning model. Here, a process in a learning process of performing specialized learning, which is a target of the present invention, will be mainly described.

First, the medical image group conversion unit 21 acquires a large number of medical images (first image group) from the storage device 40 according to an input from the system or an instruction from a user (step 101), and generates a plurality of image groups (second image groups) on the basis of a plurality of (1 to n) conventional features (first features). The conventional features F1 to Fn are determined in advance and are, for example, the circularity of a lesion tissue, the distance between an organ and a tumor, a degree (hereinafter, it is referred to as degree of spiculation) of a spicula of the tumor contour, and the like, and are held in the medical image group conversion unit 21 in the form of a table together with a corresponding feature calculation formula, for example.

Next, the medical image group conversion unit 21 calculates values of the respective conventional features for the respective images constituting the first image group by using the held feature calculation formula, and creates criteria for the creation of an image group (step 102). Specifically, thresholds regarding the conventional features are set as the creation criteria. A plurality of image groups (second image groups) are generated based on the values of the conventional features of each image and the thresholds (step 103). The second image groups are not obtained by clustering the input image group into a plurality of images, but are image groups newly formed using the thresholds for the conventional features, and one image included in the input image group may be included in two or more new image groups. A specific method of setting the image group creation criteria will be described in an embodiment to be described later. By setting the image group creation criteria in the learning process, it is possible to extract a deep learning feature specialized for a conventional feature with respect to a target image in the subsequent operation process.

Next, the feature extraction unit 22 calculates new features (second features) for the second image groups (step 104). The processing from step 102 to step 104 is performed on all the conventional features (F1 to Fn). In this manner, since the processing is performed on the new features for each of the image groups generated on the basis of the conventional features, the new features are features reflecting information of the conventional features. Next, the feature integration unit 23 integrates the new features for each image group and outputs an integrated feature (step 105).

The lesion-related information prediction unit 24 learns the relevance between the integrated feature and lesion-related information (for example, the malignancy of the lesion region of the medical image group) and generates a prediction model (step 106). The generation (training) of the prediction model is similar to learning using a normal CNN or the like, and the training is mainly performed using teacher data.

The above is the processing in the learning stage.

In operation, an image to be diagnosed is input via the input device 10, and the medical image group conversion unit 21 calculates a value of each conventional feature according to a feature calculation formula set in advance for the target image. It is assumed that the target image belongs to a plurality of image groups based on the calculated values of the features. Which image group the target image belongs to is determined on the basis of the creation criteria (thresholds) set in the medical image group creation unit 21, and there is a possibility that one target image belongs to one or more image groups.

Thereafter, a new feature is extracted for each image group by the feature extraction unit 22 (any one of feature extraction units 1 to n) trained specifically for each conventional feature. The feature integration unit 23 integrates the new features for each image group, and inputs the integrated feature to the lesion-related information prediction unit 24. The lesion-related information prediction unit 24 outputs, to the monitor, a prediction result, which is an output thereof.

In the medical image processing system 100, since it is possible to perform feature extraction reflecting a conventional feature, that is, findings of an expert such as a doctor, it is possible to obtain a prediction result with high understanding of the expert and high accuracy.

The above-described configurations and functions of the image processing apparatus 20 can be implemented by software by, for example, a computer including a memory and a CPU or a GPU interpreting and executing a program for implementing each function. In addition, a part or all of each configuration and function of the image processing apparatus 20 may be implemented by hardware, for example, by designing with an integrated circuit. Information such as a program, a table, and a file for implementing each function can be stored in a storage device such as a memory, a hard disk, or a solid state drive (SSD), or a recording medium such as an IC card, an SD card, or a DVD.

Note that, in FIG. 1, control lines and information lines considered to be necessary for description are illustrated, and not all control lines and information lines are necessarily illustrated. In practice, it may be considered that almost all the configurations are connected to each other.

Furthermore, FIG. 1 illustrates a case where the image processing apparatus 20 is an element of the medical image processing system 100. However, the image processing apparatus according to the present embodiment may be attached to a medical imaging device 50 such as an MRI apparatus, a CT apparatus, or an X-ray imaging apparatus, or may be a part of the medical imaging device 50, or may be connected to one or a plurality of medical imaging apparatuses.

Next, a specific embodiment of each unit of the image processing apparatus will be described based on the above-described embodiment of the image processing apparatus. In the following embodiment, a basic configuration and operation are similar to those illustrated in FIGS. 1 to 3, and these drawings are referred to as necessary.

### First Embodiment

An image processing apparatus 20 according to the present embodiment includes a medical image group conversion unit 21, a feature extraction unit 22, a feature integration unit 23, and a lesion-related information prediction unit (hereinafter, simply referred to as a prediction unit.) 24, similarly to the configuration illustrated in FIG. 1. As illustrated in FIG. 2(A), the medical image group conversion unit 21 further includes a feature calculation unit 211 that calculates a first feature and a threshold calculation unit 212 that calculates a threshold for the feature. The feature extraction unit 22 and the prediction unit 24 include a multilayer neural network such as CNN.

Hereinafter, details of processing (each step in FIG. 3) in a learning process of the image processing apparatus 20 according to the present embodiment will be described with reference to FIGS. 3 to 5.

### [Step 101]

An image group P0 including a large number of images p1 to pm (represented as pc. c=any of 1 to m) stored in the storage device 40 is input via the input device 10.

### [Step 102, Step 103]

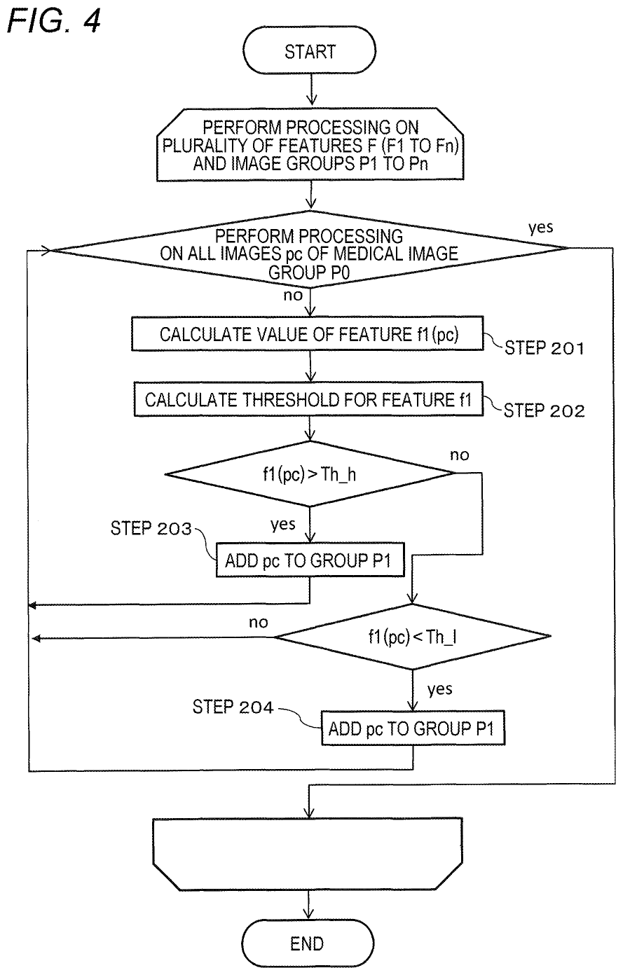

The medical image group conversion unit 21 creates image groups P1 to Pn regarding conventional features from the image group P0. The processing of the medical image group conversion unit 21 will be described with reference to FIG. 4.

First, for all the images included in the image group P0, a value of a feature is calculated according to a calculation formula f (pc) for a predetermined conventional feature F (S201). Examples of the conventional feature F include circularity of a lesion site (for example, a tumor), a distance between a specific organ and the tumor, and a degree of spiculation. For each of the circularity and the distance, a calculation formula for calculating a value of the feature using a measurement value automatically or manually measured is defined.

However, the conventional feature used in the present embodiment may be a feature not expressed by a calculation formula, in addition to the expressed feature obtained by the calculation formula.

For example, as described above, it is difficult to quantify the “degree of spiculation” only from an image, but in the present embodiment, one or a combination of the following features is used as the “degree of spiculation”. First, the frequency (contour frequency) is calculated from the amplitude of the contour shape of the tumor. The contour frequency is considered to have a relatively high correlation with the degree of spiculation. However, the contour frequency is not a complete numerical representation of the degree of spiculation, and is a numerical representation of a part of the knowledge of a doctor, but is not the same as a feature that is captured as the degree of spiculation when interpreted by the doctor.

In the second example, for each piece of learning data (image), a value obtained by evaluating the degree of spiculation on a scale of 10 (1 to 10) by the doctor is defined as a feature (subjective feature). Since the subjective feature is a value given by the doctor himself/herself, it can be said that the subjective feature is a value representing knowledge in terms of an order scale (a scale having meaning in order and magnitude). However, since the evaluation is based on subjectivity, it is assumed that there is variation in accuracy in terms of an interval scale (scale in which graduations are equally spaced and the interval is meaningful) and the like.

Therefore, these features can be combined, or weighted and combined as necessary, to obtain a feature representing the “degree of spiculation”.

Next, for each of the features F1 to Fn, thresholds (upper limit and lower limit) Th_h and Th_l are set (S202), the value f1(p) of the feature calculated in S201 is compared with the thresholds, and when the value is larger than the upper limit Th_h or smaller than the lower limit Th_l, this image is added to the image group P1 (S203, S204).

For the setting of the thresholds (S202, FIG. 3: 102), appropriate values can be determined in advance on the basis of an experience value or the like at the time of designing the image processing apparatus. However, usually, information of an actual input image cannot be obtained at the time of designing, and it is considered that the distribution of an actually input image group is different from an assumption at the time of designing in many cases. Therefore, in the present embodiment, the thresholds are evaluated using a discriminator, and are set so as to have a high identification ability. A specific method will be described later.

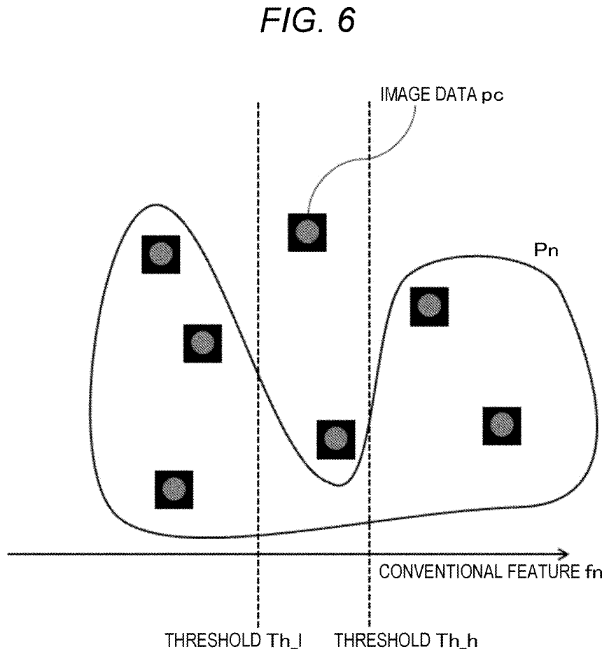

By executing the above-described S203 to S205 for all the images p1 to pm, the image to be added to the image group P1 is determined. As a result, the one image group P1 regarding the conventional feature F1 is generated. This state is illustrated in FIG. 6. In FIG. 6, the horizontal axis represents the value of the conventional feature F1. A set of a plurality of (here, seven) images illustrated in the drawing is the image group P0. Images enclosed by a curve, three images having a value of a feature smaller than the threshold (lower limit value) Th_l, and two images having a value of a feature larger than the upper limit Th_h are the second medical image group P1.

Similarly, for the conventional features F2 to Fn, addition to the image groups P2 to Pn regarding the conventional features F2 to Fn is performed on all the images of the image group P0, and finally, image groups P1 to Pn corresponding to all the conventional features are generated (repeating step 200).

Next, an example of a method of setting the thresholds Th_h and Th_l which are the medical image group creation criteria (step 202) will be described.

As described above, in a case where the thresholds are set to predetermined values in advance, appropriate image group generation cannot be performed depending on the distribution of the input image group. For example, in a case where the thresholds are set as fixed values at the time of design, the number of pieces of image data P_num included in the image groups P1 to Pn may be extremely small (may be 0 in some cases), and the subsequent processing may not be executed, depending on the distribution of the input image group. On the other hand, a method is also conceivable in which the minimum value N_min of the number of pieces of the image data included in the image group is determined in advance, and pieces of the image data are collected in order from the smallest value and the largest value of f0 in the input image group until P_num≥N_min is satisfied. In this case, it is guaranteed that the generated image groups have sizes enough to withstand the subsequent processing, but there is a possibility that most (all in some cases) of the input image group P0 becomes one image group P. In this case, the original purpose of combining a group having a large feature and a group having a small feature into one group is not achieved.

In order to avoid such a situation, in the present embodiment, first, an image group Px is used as an input, a discriminator (for example, a multilayer neural network trained to predict a malignancy associated with a lesion appearing in each image pc′) that predicts information included in image data pc′ belonging to an image group Py is created. In this case, if the thresholds Th_l and Th_h used to generate the image group Py are changed without changing the configuration of the discriminator, it is assumed that the identification accuracy of the discriminator changes. The thresholds used when the image group that can be identified best using the discriminator is generated are set as the final thresholds Th_l and Th_h. Note that it is preferable to set a minimum value to the total number of images pc′ used when the thresholds are determined using such a discriminator.

In a case where such a method is used, even if there is a bias in the distribution of the input images with respect to each value of the conventional features F1 to Fn, it is possible to execute appropriate specialized learning regardless of the distribution. In addition, in a case where only a group having a large value of a specific conventional feature and a group having a small value of a specific conventional feature are set as a second medical image group, a feature specialized for a difference between the values of the conventional features can be extracted in the next feature extraction processing.

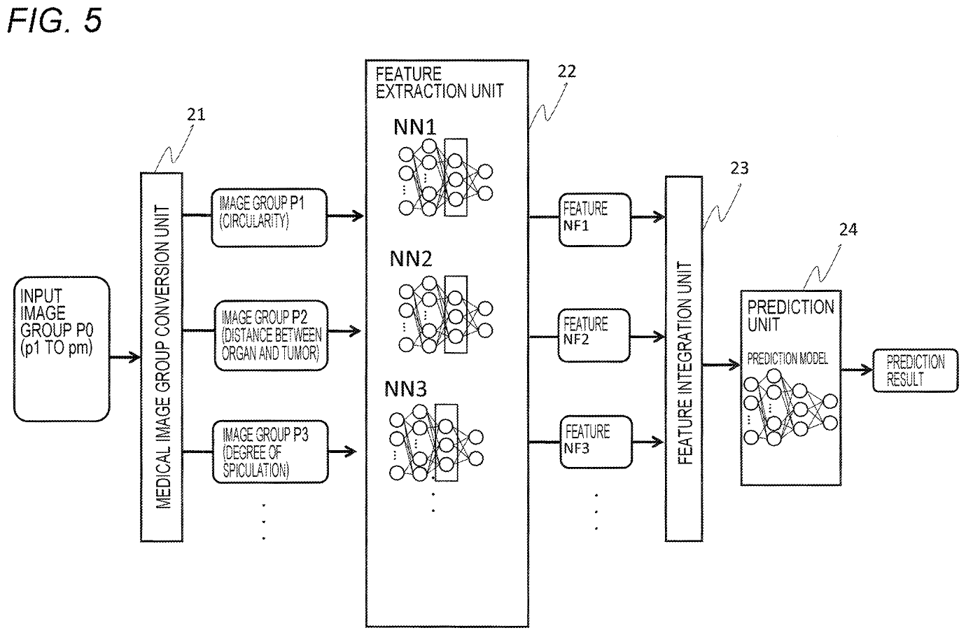

With the above-described steps 200 to 204, the image group conversion unit 21 can generate the image groups P1 to Pn specialized for the features as illustrated in FIG. 5.

### [Step 104]

The feature extraction unit 22 extracts a new feature (second feature) by deep learning for each of the plurality of image groups generated in step 103. For the extraction of the new features, for example, an intermediate output of the multilayer neural network in a case where learning for predicting the malignancy associated with the lesion of the second medical image group is performed using the second medical image group as an input may be used, or a feature generation network using an auto encoder may be adopted. The auto encoder generally refers to an algorithm for dimension compression using a neural network, and is obtained by performing supervised learning using the same data for an input layer and an output layer in a neural network of three or more layers. This is also considered as a method of compressing the dimension of the input data, and the output of an intermediate layer here can also be referred to as a feature representation obtained by dimensionally compressing the input data.

The setting of the network configuration in the feature extraction unit 22 may be performed on the basis of the accuracy of regression prediction by machine learning or the like, or may be performed on the basis of, for example, a result of correlation analysis with lesion information, or specialized knowledge regarding data or a final predicted event.

For example, in a case where the correlation between a conventional feature and lesion information to be finally predicted is very high, it is considered that there is a higher possibility that an appropriate new feature can be calculated in a case where the lesion information is used as an objective variable. However, depending on the input image group, a conventional feature having a low correlation with the lesion information may be present. That is, even if a doctor has knowledge that the correlation between the feature and the lesion information is high, the correlation between the feature and the lesion information may not be high only in the input image group that can be acquired due to a bias of a subject group or the like. In such a case, there is a high possibility that an auto encoder intended to accurately capture the feature of the input image group can calculate an appropriate DL feature, rather than supervised learning using the lesion information as an objective variable. Whether the accuracy of the final prediction result is increased in the case where the output of the intermediate layer of the feature generation network is set to the new feature or in the case where the intermediate output of the network in which the malignancy is learned is set to the new feature may be checked by experiment, and the configuration in the case where the accuracy is high may be adopted.

In addition, the configuration of the neural network may be the same for each image group, but a different feature generation network may be configured according to the type of a conventional feature.

The feature extraction unit 22 calculates new features (NF1 to NFn) for each of the image groups P1 to Pn by the network set for each image group as described above.

### [Step 105]

The feature integration unit 23 integrates the new features (second features) calculated in step 104 to calculate an integrated image feature. As the integration method, a union of the new features may be simply used as the integrated image feature, or the integration may be performed after a feature selection process of selecting and using a combination of valid features from the new features (NF1 to NFn) is performed.

The feature selection process is a process of searching for a combination of features effective when a machine learning model is used. In a case where similar image features are included in the features to be integrated, if a simple union is used as an integrated image feature, there is a possibility of causing over-learning, but the possibility of over-learning can be avoided by adding this process.

### [Step 106]

The prediction unit 24 learns the relevance between the integrated image feature and the lesion-related information (for example, the malignancy of the lesion region of the medical image group) and generates a prediction model. The generation (training) of the prediction model is similar to training using a normal CNN or the like, and the training is mainly performed using teacher data.

Although the processing in the learning process of the image processing apparatus of the first embodiment has been described above, the operation is similar to that described above.

According to the present embodiment, it is possible to acquire an appropriate deep learning feature according to a conventional feature, and it is possible to perform specialized learning with higher accuracy. In addition, according to the present embodiment, the contour frequency or the subjective feature is used as an axis for capturing the degree of spiculation on an image, and deep learning is performed along the axis, so that it is possible to perform specialized learning even for a conventional feature that has been difficult to extract, and is, for example, the “degree of spiculation”, and it is possible to output a prediction result contributing to diagnosis.

<Modification 1 of Embodiment>

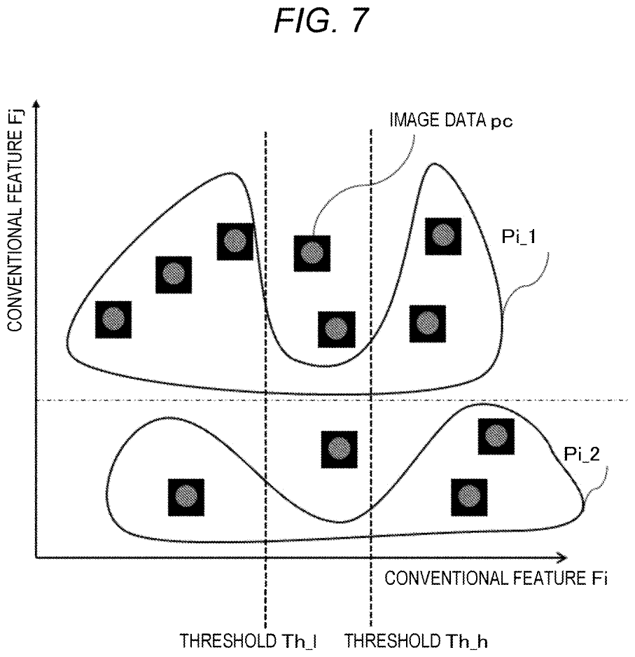

As processing of the medical image group conversion unit 21, in the example illustrated in FIG. 6, one image group is created using one conventional feature, but it is also possible to generate an image group with reference to a value of another conventional feature other than the conventional feature. For example, FIG. 7 illustrates a set of input image groups, the horizontal axis represents a conventional feature Fi (i=any one of 1 to n), and the vertical axis represents another conventional feature Fj (j=any of 1 to n, provided that j≠i). As illustrated in the image, the group divided by the thresholds Th_l and Th_h for the conventional feature Fi is divided into two sets, a set P1_1 having a large value of Fj and a set P1_2 having a small value of Fj in consideration of the values of the other features Fj. That is, a plurality of image groups are generated as the second image group. In this case, a set (here, P1_1) in which a distribution of the values fi of the features is the best balanced is set as the image group Pi for the feature Fi.

As described above, to generate a second image group (P1) regarding one conventional feature (for example, F1), another conventional feature other than the conventional feature is used, and a group in which only similar features are collected with respect to features other than the conventional feature F1 can be used as an input, so that a feature specialized for the feature F1 can be extracted.

### Second Embodiment

In the first embodiment, the feature extraction unit 22 calculates the features of the second image groups generated in association with the conventional features. However, in the present embodiment, in addition to the extraction of the features of the second image groups, the feature extraction unit 22 extracts a feature of an input image group and uses the extracted feature for calculation of an integrated image feature.

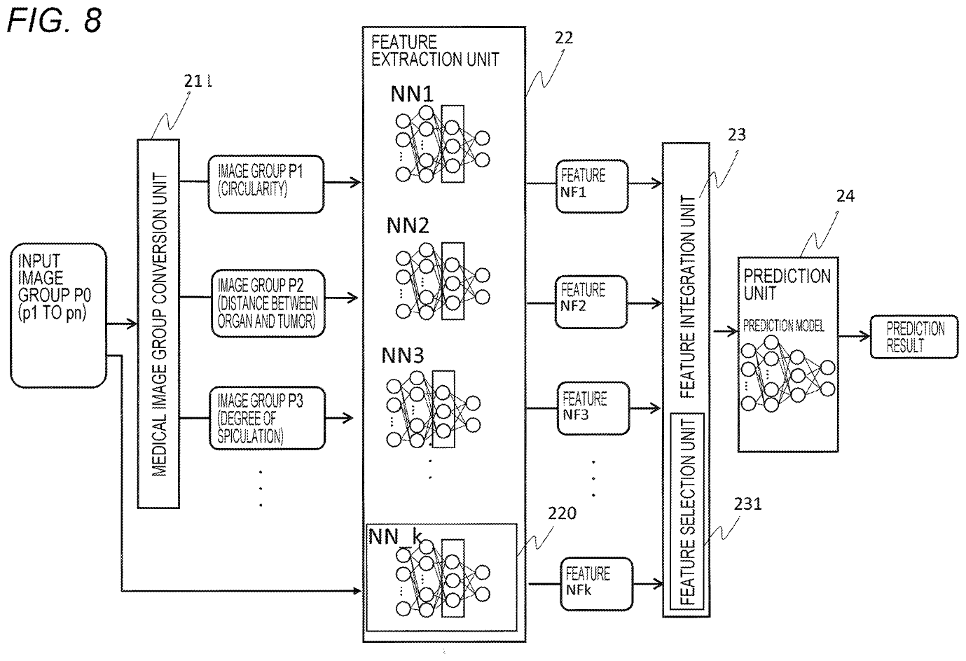

FIG. 8 illustrates processing according to the present embodiment. In FIG. 8, the same elements and processes as those in FIG. 5 are denoted by the same reference numerals, and differences from the first embodiment will be described below.

In the present embodiment, the feature extraction unit 22 includes a feature extraction unit (second feature extraction unit) 220 that receives an input image group, which has not passed through the medical image group conversion unit 21, and extracts a feature thereof, in addition to a plurality of feature extraction units (FIG. 2 (B)) that extract features of a plurality of image groups regarding conventional features. The feature extraction unit 220 calculates a deep learning feature NFk for an image group Pk equal to the input image group. In this case, for example, a feature generation network using an auto encoder can be used for the feature extraction. Although the extracted feature NFk is not a new feature obtained by learning specialized for the conventional features, it is considered that the extracted feature NFk includes a feature that cannot be extracted by the specialized learning for the conventional features, and it is expected that the accuracy of prediction is improved by adding the feature NFk to the next feature integration.

The feature integration unit 23 integrates the new features NF1 to NFn of the image groups P1 to Pn extracted by the feature extraction unit 22 and the feature NFk of the image group Pk. Here, there is a possibility that image features similar to the new features NF1 to NFn extracted by the specialized learning may also be extracted as the feature NFk. Therefore, in the present embodiment, after a feature selection process (process of a feature selection unit 231 in FIG. 8) is performed, the features are integrated.

As the feature selection process, a process of deleting a feature that does not affect an objective variable (feature selection process 1), a process of deleting a feature that shows the same tendency (for example, when each of Fx and Fy is treated as one feature, there is a relationship of Fy=Fx+a) (feature selection process 2), a process of deleting a feature having a very high correlation (feature selection process 3), and the like are known. These processes can also be used in the present embodiment, but conventionally, the feature selection processes 2 and 3 are performed on all combinations of candidate features, whereas in the present embodiment, the feature selection process is performed between the feature NFk (feature obtained by learning the original image) and each of the new features NF1 to NFn (new features calculated for the image groups P1 to Pn).

Since it is assumed that the second image features along different conventional features are calculated for NF1 to NFn, there is a low possibility that independent and similar features are calculated in combinations of these, but there is a possibility that a feature similar to the feature Fk obtained by learning of the original image is present. Therefore, by performing the feature selection process as described above, overfitting can be avoided, and improvement in the accuracy of the prediction can be expected.

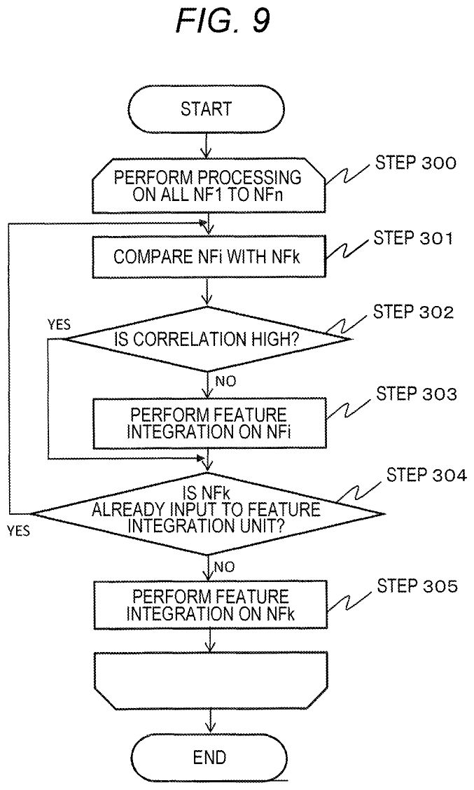

FIG. 9 illustrates an example of an integration process including the feature selection process according to the present embodiment. In this example, one new feature (for example, NF1) is compared with NFk (step 301), and when the correlation is not high, NF1 is used as an input of the feature integration unit 23 (step 302, step 303). When the correlation between the two is high, NF1 is deleted, and NFk is input to the feature integration unit 23 (step 305) on condition that NFk has not been input to the feature integration unit 23 (step 304). The steps 301 to 305 are executed on all the new features NF1 to NFn (step 300). Through such processing, a feature that has a high correlation with NFk is excluded, and the prediction unit 24 generates an integrated feature for performing effective predictive learning.

Here, each of the features Fk and NF1 to NFn may be a feature group including a plurality of features. For example, when the new feature group NF1 is described, each feature of NF1 and each feature of Fk are compared in a brute-force manner in step 301, and the processing is performed on each feature belonging to NF1 in steps 302 and 303. That is, among the features belonging to NF1, some features having a high correlation with NFk are deleted, and some other features are input to the feature integration unit 23.

Note that FIG. 9 illustrates an example in which the feature selection process 3 (selection using correlation) is adopted as a method of the feature selection process. However, the feature selection process 2 can be similarly performed by replacing the determination step 302 with processing of determining “whether features exhibit the same tendency”.

According to the present embodiment, there is a possibility that the accuracy of the prediction is improved by adding a feature extracted from the original image. In addition, the possibility of over-learning can be reduced by performing the selection process at the time of the feature integration.

<Modification 2>

In the first and second embodiments, the case where the image features are mainly used as the conventional features has been described, but the conventional features are not limited to the image features. For example, it is also possible to perform specialized learning using, as a value of a conventional feature, a value of a blood tumor marker for a tumor as a target. In this case, not only a medical image but also, for example, a blood test result, genomic variation information, and the like are input as input data of the image processing apparatus 200, a feature of the input data is extracted by the feature extraction unit 22 (second feature extraction unit 220), and the feature integration unit 23 calculates an integrated feature together with a DL feature for each image group.

According to the present modification, not only an image but also a plurality of types of patient information such as a blood test and genomic variation information can be integrally captured, and in a case where the present modification is applied to diagnosis, treatment, and the like of a tumor, more accurate information can be provided, and high contribution can be expected.

Although the embodiments and the modifications of the present invention have been described above, the present invention is not limited to the above-described embodiments and modifications, and includes various modifications. For example, the above-described embodiments have been described in detail for easy understanding of the present invention, and are not necessarily limited to those including all the described configurations. In addition, a part of the configuration of a certain embodiment can be replaced with the configuration of another embodiment, and the configuration of a certain embodiment can be added to the configuration of another embodiment. In addition, for a part of the configuration of each embodiment, it is possible to add, delete, and replace another configuration.

### REFERENCE SIGNS LIST

10 input device
20 medical image processing apparatus
21 medical image group conversion unit
22 feature extraction unit
23 feature integration unit
24 lesion-related information prediction unit
30 monitor
40 medical image storage unit

1. An image processing apparatus that processes a medical image, the image processing apparatus comprising:
an image group conversion unit that calculates a value of a predetermined feature for each image constituting an input first image group, selects an image from the first image group on the basis of the value of the feature, and sets the image as an image of a second image group; and
a feature extraction unit that performs learning on the second image group generated by the image group conversion unit using a feature generation network and extracts a new feature.

2. The image processing apparatus according to claim 1, wherein
the image group conversion unit generates a machine learning discriminator for the predetermined feature, evaluates identification performance of the discriminator, and sets a feature threshold for selecting an image based on the identification performance.

3. The image processing apparatus according to claim 1, wherein
the predetermined feature includes a plurality of features, and
the image group conversion unit selects an image based on a value of each of the plurality of features and generates a second image group for each of the plurality of features.

4. The image processing apparatus according to claim 3, wherein
the feature extraction unit has a network structure having configurations different for the plurality of features.

5. The image processing apparatus according to claim 3, further comprising a feature integration unit that integrates new features extracted by the feature extraction unit for each of a plurality of second image groups.

6. The image processing apparatus according to claim 1, wherein
the feature extraction unit includes a second feature extraction unit that extracts a feature of input patient information, and
a feature integration unit that integrates the new feature extracted for the second image group by the feature extraction unit and the feature extracted by the second feature extraction unit.

7. The image processing apparatus according to claim 6, wherein
the second feature extraction unit receives the first image group as the patient information and extracts features of the first image group.

8. The image processing apparatus according to claim 7, wherein
the feature integration unit compares the features of the first image group with the new feature, and includes a feature selection unit that excludes a redundant feature.

9. The image processing apparatus according to claim 6, wherein
the second feature extraction unit receives information other than an image as the patient information.

10. The image processing apparatus according to claim 1, wherein
the predetermined feature includes a degree of a spicula of a contour of a tumor.

11. The image processing apparatus according to claim 10, wherein
the feature extraction unit uses, as a feature of the degree of the spicula of the contour of the tumor, at least one of a frequency calculated from an amplitude of the contour shape and a grade evaluation by an expert.

12. The image processing apparatus according to claim 1, further comprising a prediction unit that receives the new feature and outputs a prediction result regarding a lesion.

13. An image processing method comprising:
a step of inputting a first image group and calculating a value of a predetermined feature for each image constituting an input first image group;
a step of setting a threshold for the feature;
an image group conversion step of selecting an image from the first image group on the basis of the value of the feature and setting the image as an image of a second image group; and
a step of extracting a new feature by performing learning on the second image group using a feature generation network, wherein
in the step of setting the threshold, a machine learning discriminator is generated for the predetermined feature, identification performance of the discriminator is evaluated, and the feature threshold for selecting an image is set based on the identification performance.

14. The image processing method according to claim 13, wherein
the predetermined feature includes a plurality of features, and
the image group conversion step generates a plurality of the second image groups for the plurality of features, and
the step of extracting the new feature extracts a new feature for each of the plurality of second image groups, and
the image processing method further comprises a step of integrating the plurality of new features.

15. An image processing program for causing a computer to execute:
a step of inputting a first image group and calculating a value of a predetermined feature for each image constituting the input first image group;
a step of setting a threshold for the feature;
an image group conversion step of selecting an image from the first image group based on the value of the feature and setting the image as an image of a second image group; and
a step of extracting a new feature by performing learning on the second image group using a feature generation network.
